(19) United States
(12) Patent Application Publication
(10) Pub. No.: US 2004/0126431 A1
(43) Pub. Date: Jul. 1, 2004

(54) Method for preparing microspheres containing a water-soluble substance

(76) Inventors: Frederic Lagarce, Angers (FR); Odile Cruaud, Avrille (FR); Jean-Pierre Benoit, Avrille (FR); Christine Deuschel, Trelex (CH)

Correspondence Address: NIXON & VANDERHYE, PC, 1100 N GLEBE ROAD, 8TH FLOOR, ARLINGTON, VA, 22201-4714, US

(21) Appl. No.: 10/398,024

(22) PCT Filed: Oct. 3, 2001
(86) PCT No.: PCT/CH01/00596

(30) Foreign Application Priority Data
Oct. 3, 2000 (CH) 1941/00

(51) Int. Cl. A61K 9/16; A61K 9/50; B01J 13/02; B01J 13/04

(52) U.S. Cl. 424/490; 264/4.1

(57) ABSTRACT

The invention concerns a novel method for preparing, by solvent extraction, PLGA/PLA-type polymer microspheres encapsulating water-soluble substances. The invention also concerns the microspheres obtained by said method.

The present invention relates to a novel method for preparing, by solvent extraction, poly(D,L-lactide-co-glycolide)/poly(D,L-lactide) (PLGA/PLA)-type polymer microspheres encapsulating a water-soluble therapeutic substance which is not soluble in ethyl acetate, and to the microspheres which can be obtained using this method.

The microencapsulation of therapeutic substances in microspheres made of biodegradable PLGA/PLA polymer are of great value in preparing delayed-release formulations. Such formulations increase the effectiveness of treatment with cytostatic anticancer agents since they make it possible, by intraperitoneal (i.p.), intratumor (i.t.) or intrapleural injection, to increase local concentration while at the same time decreasing systemic toxicity. For easy injection without any risk of clogging of the needles conventionally used, the microspheres should have an average size of 30-100 μm, preferably 20 to 70 μm.

Hongkee Sah, 1997, Journal of Controlled Release, 233-245, describes the preparation of PLGA microspheres using a method by solvent extraction. This method consists in pouring an aqueous solution of polyvinyl alcohol into a solution of ethyl acetate containing this polymer, with stirring, so as to form an emulsion, and then in extracting the ethyl acetate by adding distilled water which induces microsphere formation, retaining and washing the microspheres by filtration, resuspending them in a solution of polyvinyl alcohol and, finally lyophilizing them. That document studies the influence of the ratio of the volume of ethyl acetate to the volume of polyvinyl alcohol solution on the size and the structure of the microspheres obtained.

U.S. Pat. No. 5,238,714 describes the preparation of PLGA polymer microspheres encapsulating cisplatin using a method by solvent evaporation. This method consists in dispersing particles of cisplatin in methylene chloride, adding PLGA to this solution, forming an emulsion by mixing, with vigorous stirring, the organic phase with approximately 6 times greater volume of aqueous polyvinyl alcohol solution comprising 0.9% of NaCl, the pH of which has been adjusted to less than 4 by adding HCl, then evaporating the methylene chloride for several hours, which induces microsphere formation, retaining and washing the microspheres by filtration and, finally, air-drying them. The PLGA microspheres encapsulating the cisplatin obtained have, depending on the operating conditions, a diameter of approximately 100 μm or of 1-5 μm.

The method disclosed in the above patent is not suitable for water-soluble therapeutic substances such as oxaliplatin since these substances pass at least partly into the aqueous phase during evaporation of the organic phase and there is therefore very little, if any, encapsulation thereof in the microspheres. The microspheres therefore have a low degree of encapsulation which is not acceptable for a delay-formulation. This formulation, intended to be injected, should in fact release a given amount of therapeutic substance without having too greater volume. In addition, this method uses a saline solution and HCl, substances which react with oxaliplatin.

The problem of the invention is therefore to find a method for preparing PLGA microspheres with an average size of 20-100 μm encapsulating a water-soluble therapeutic substance, in particular oxaliplatin, with an acceptable degree of encapsulation, for example of at least 10%, and continuous release for a period of therapeutic interest.

This problem is solved by the invention as defined in the claims.

The method of the invention makes it possible to obtain PLGA/PLA microspheres of 20 to 100 μm with a degree of encapsulation of water-soluble therapeutic substances of 10 to 45%, an encapsulation yield of at least 80% and a production yield of at least 80%, with continuous release of this substance for 5 to 60 days. This corresponds to a period of release of therapeutic interest for a cytostatic such as oxaliplatin.

The invention relates to a method for preparing, by solvent extraction, poly(D,L-lactitde-co-glycolide)/poly(D,L-lactide) (PLGA/PLA)-type polymer microspheres encapsulating a water-soluble therapeutic substance which is not soluble in ethyl acetate, comprising the following steps:

(a) preparing an organic phase comprising dissolved polymer and particles of the therapeutic substance in suspension in ethyl acetate, and cooling this organic phase so as to increase its viscosity,

(b) adding to this organic phase a cold aqueous solution of surfactant with vigorous stirring so as to form an emulsion,

(c) extracting the ethyl acetate by adding hot water, with stirring to the emulsion kept at low temperature, which induces microsphere formation,

(d) filtering the heterogeneous mixture obtained in (c) so as to retain the microspheres formed and rinsing so as to remove the residual surfactant and, optionally,

(e) lyophilizing the microspheres.

Step (a) can be carried out either by adding the polymer to a suspension of particles of the therapeutic substance in ethyl acetate, with stirring, or by adding particles of the therapeutic substance to a solution of the polymer in ethyl acetate, with stirring.

The size of the particles of the therapeutic substance should be very much less than the desired size of the microspheres. For the desired size range of the microspheres, from 20 to 100 μm, a therapeutic substance particle size of less than 1 μm is very suitable. The particles of the therapeutic substance of desired size can, for example, be obtained by grinding this substance in a centrifugal ball mill, possibly in the presence of the organic phase.

The therapeutic substance should be water-soluble and not soluble in ethyl acetate. The expression “solubility in water” is intended to mean a solubility at ambient temperature (25° C.) of at least 3 g/l, preferably of at least 5 g/l, and the expression “insolubility in ethyl acetate” is intended to mean a solubility at ambient temperature of less than 10 mg/l, preferably 1 mg/l. The therapeutic substances which are advantageous for delayed-release formulations are cytostatic anticancer agents. An example of such substance is, for example, oxaliplatin.

It is essential to control the temperature during the various phases of this method in order to obtain the desired advantages.

The organic phase prepared during step (a) should be cooled in order to increase the viscosity of this phase, preferably to a temperature sufficiently low to avoid sedimentation of the therapeutic substance, generating a decrease in the encapsulation yield (defined as the ratio of the real degree of encapsulation to the theoretical degree of encapsulation if all the therapeutic substance used was in the microspheres). This temperature is preferably from 6 to −20° C., in particular 3 to 0° C.

The temperature of the cold aqueous solution of surfactant, which regulates the viscosity of the dispersing phase and therefore the size of the micro-particles, should be sufficiently low to obtain the desired size range of 20 to 100 μm, and to limit the solubilization of the therapeutic substance in this phase. This temperature is preferably from 14 to 2° C., in particular 6 to 8° C.

During step (b), the emulsion being formed is generally maintained at a temperature of below 8° C., preferably below 5° C., by adequate cooling.

During step (c), the emulsion being extracted is maintained at low temperature, i.e. at a temperature at least 20° C. below the temperature of the hot water added. This low temperature is preferably below 8° C., in particular below 5° C.

The temperature of the hot water added during step (c) should be sufficiently high to avoid too rapid an extraction of the ethyl acetate with the water. The solubility of ethyl acetate in water in fact decreases with an increase in temperature. This temperature should be below the glass transition temperature of the polymer used. This temperature is preferably from 26 to 40° C., in particular 28 to 35° C.

The ratio of the volume of ethyl acetate used to prepare the organic phase to the volume of cold aqueous solution of surfactant should be sufficiently low so that the proportion of ethyl acetate which leaks into the water does not induce microsphere formation by precipitation of the polymer. This ratio is preferably 0.10-0.20, in particular 0.12-0.18.

The volume of ethyl acetate used to prepare the organic phase is preferably 8-16 ml, in particular 10-14 ml per g of polymer.

The volume of water added during step (c) is a minimum of approximately 10 times that of ethyl acetate, preferably at least 20 times this volume.

In order to control the rate of formation of the microspheres, to decrease the production waste and therefore to increase the production yield (defined as the ratio of the mass of the lyophilized microspheres to the sum of the mass of the polymer and of that of the therapeutic substance), it is preferable to add the hot water gradually. For example, the volume of water added during step (c) can be divided up into n successive additions of 1/n of this volume, n being between 2 and 10, for periods of 30 sec to 5 minutes.

The surfactant is preferably a nonionic surfactant, such as, for example, a polyvinyl alcohol (PVA).

The poly(D,L-lactide-co-glycolide)/poly(D,L-lactide) (PLGA/PLA)-type polymer here denotes either a copolymer of D,L-lactic acid and glycolic acid (PLGA), or a mixture of one or more copolymers of D,L-lactic acid and glycolic acid and/or one or more polymers of D,L-lactic acid(PLA), this mixture generally consisting of 80 to 100% of PLGA and of 0 to 20% of PLA.

The composition of the PLGA, and in particular the respective percentages of lactic acid units and glycolic acid units, are chosen according to the desired in vivo degradation kinetic for this polymer, which have an influence on the release of the therapeutic substance. For a therapeutic substance such as oxaliplatin, a PLGA comprising 25 D-lactic acid units and 25 L-lactic acid units per 50 glycolic acid units (PLGA 25/50) is preferred.

The molecular weight of the PLGA should not be too high so as to allow its solubilization in the ethyl acetate. This molecular weight is preferably less than 50 000, in particular between 10 000 and 40 000.

The PLA preferably has a molecular mass of 1000 to 5000, in particular 1500 to 3000.

The invention also relates to the novel PLGA/PLA microspheres encapsulating a therapeutic substance, in particular oxaliplatin, which can be obtained using the method described above.

These microspheres preferably have a degree of encapsulation of the therapeutic substance of at least 10%, in particular of 15 to 40%, and advantageously an average size of 20-100 μm, in particular of 30-70 μm.

These microspheres have the advantage of releasing 100% of the therapeutic substance continuously over 5 to 60 days with a burst at 24 hours of 15 to 45%. This corresponds to a period of release of therapeutic interest for a cytostatic such as oxaliplatin.

The profile of release of the therapeutic substance, and in particular the burst at 24 hours and also the duration for the release of 100% of the substance, can be easily controlled through the choice of the degree of encapsulation, of the size of the particles and of the PLGA/PLA polymer.

A convenient way to influence the release profile through the choice of the polymer is to start with a polymer of given composition and mass, the release profile of which is known, for example PLGA 25/50 of molecular mass 40 000, and to add to it a varying proportion of another polymer of the same composition and of lower molecular mass, for example PLGA 25/50 of molecular mass 20 000 or 10 000, of a PLA oligomer, for example of molecular mass 2000 or 3000.

The microspheres can be sterilized according to conventional methods in the art, for example by irradiation with gamma rays.

The microspheres of the invention encapsulating oxaliplatin have advantageous antitumor activity.

The antitumor activity has been demonstrated in Nude mice carrying either a peritoneal ovarian adenocarcinoma of human origin after intraperitoneal (i.p.) administration, on tumors induced by the IGROV-1 cell line, or a subcutaneous prostate adenocarcinoma of human origin, after intertumor (i.t.) administration.

The microspheres of the invention encapsulating an anticancer agent such as oxaliplatin can be administered in a pharmaceutically acceptable vehicle, intraperitoneally, intertumorally or intrapleurally, and thus release the active substance locally close to the tumor or into the tumor while at the same time decreasing the diffusion of the medicinal product in the systemic circulation. The benefit is greater effectiveness of the medicinal product and greater comfort for the patient (a single injection, fewer side effects).

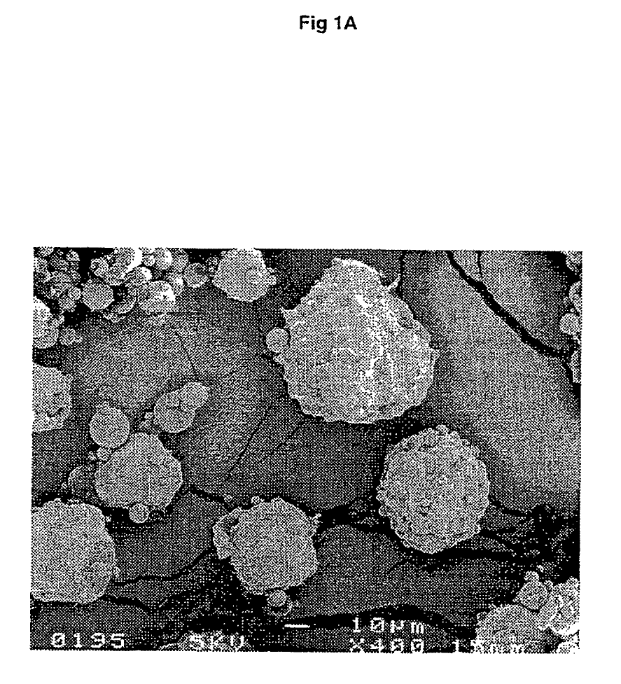

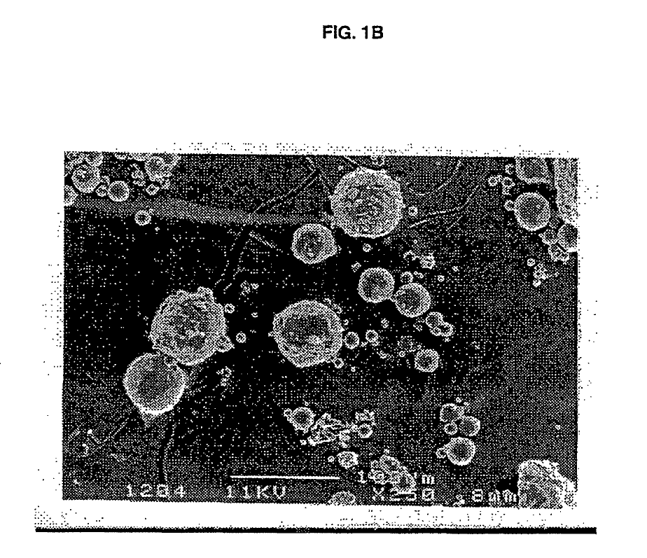

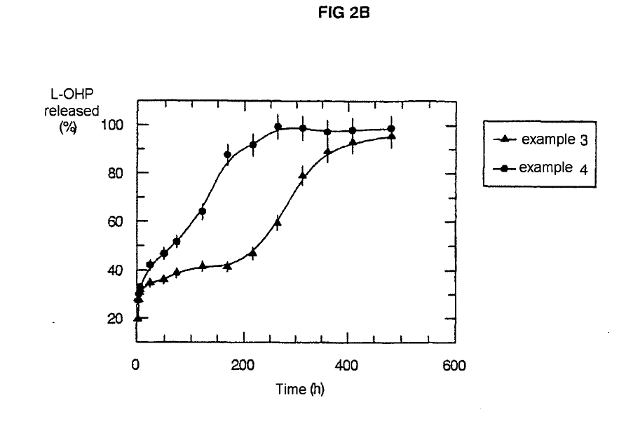

The invention therefore also relates to the microspheres defined above, for use as a medicinal product, and also a pharmaeutical formulation for i.p., i.t. or intrapleural administration comprising these microspheres in a pharmeutically acceptable vehicle. The description below will be understood more clearly by referring to FIGS. 1A, 1B, 1C, 2 and 3.

FIGS. 1A, 1B and 1C represent scanning electron microscopy photographs of the microspheres obtained in example 1 with a film-coating of gold (FIG. 1A), in example 2 with a film-coating of carbon in normal mode (FIG. 1B) and by reverse scattering (FIG. 1C).

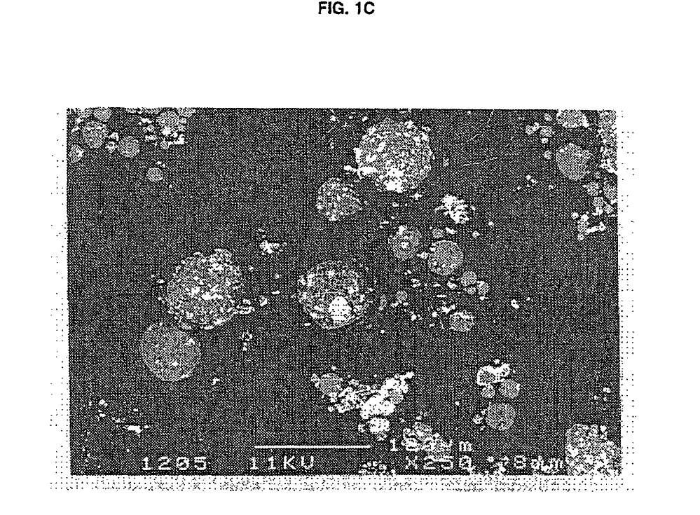

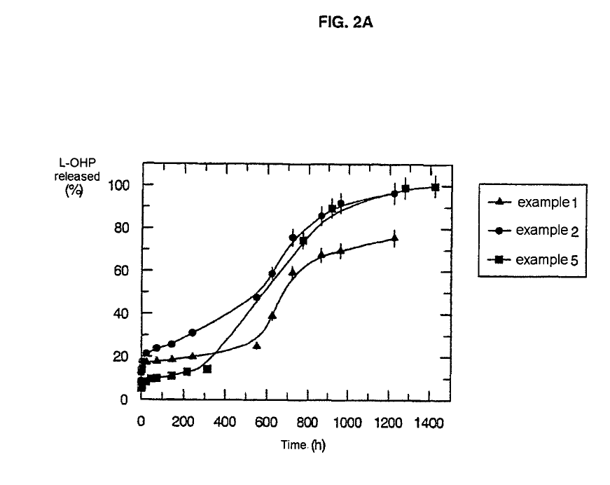

FIGS. 2A and 2B represent the oxaliplatin release curves for the microspheres obtained respectively in examples (1, 2 and 5) and (3 and 4).

The examples below illustrate the invention.

The microspheres were prepared with various copolymers of D,L-lactic acid and glycolic acid: PLGA 20/50 RG 503 of molecular mass 40 000 and PLGA 25/50 RG 502 of molecular mass 10 000 (B. I. Chimie, Saint Germain en Laye, France), PLGA 37.5/25 of molecular mass 22 000 (Phusis, Saint Ismier, France), and an oligomer of D,L lactic acid of molecular mass 2000, PLA50 (Phusis, Saint Ismier, France).

The surfactant is a polyvinyl alcohol (PVA), Rhodoviol® 4/125 88% hydrolyzed (Prolabo, Paris, France), used at 75% in water.

The filtration is carried out through 0.8 μm filters.

The oxaliplatin is ground using a centrifugal ball mill so as to obtain a powder of crystals of this substance of average size by volume 1.31 μm. The distribution is bimodal with 80% of the population centered around 0.3 μm and 20% thereof centered around 9.8 μm. The powder obtained is dispersed and ground in ethyl acetate using an Ultra-Turax disperser homogenizer (Prolabo, Paris, France), at 11 000 rpm for examples 1 to 4, and 8 000 rpm for example 5. This grinding makes it possible to obtain a suspension of particles of homogeneous size of less than 1 μm.

The size distribution of the oxaliplatin crystals and of the microspheres is analyzed using a laser granulometer (Mastersizer®, Malvern Instruments).

The microscopic observation of the microspheres is carried out on an optical microscope and a scanning electron microscope, coating said microspheres either with a film-coating of gold or with a film-coating of carbon making it possible to visualize the platinum by X-ray analysis and by reverse scattering.

### EXAMPLE 1

Preparation of Microspheres of PLGA 25/50 of Molecular Mass 40 000 with a Degree of Encapsulation of Oxaliplatin of 14%.

(F3B)

102.4 mg of oxaliplatin powder are dispersed in 6 ml of ethyl acetate at 1° C. for 3 minutes with cooling in a cryostat at 0° C. 495.5 mg of RG 503 polymer are added and dissolved with magnetic stirring at ambient temperature for 10 min, and then in a cryostat at 0° C. for 10 min. The temperature of the organic phase is 1° C.

40 ml of PVA solution at 11° C. are added to the organic phase obtained, with vigorous stirring (1500 rpm) for 2 min, so as to form an emulsion, cooling using a cryostat at 0° C.

The ethyl acetate is extracted by adding, to the emulsion in a cryostat at 0° C., over 18 min, increasing volumes of water at 32° C., 2 ml over 30 sec, 4 ml over 1 min, 8 ml over 1 min 30 sec, and 16 ml over 5 min. The emulsion is then poured into a volume of water of 440 ml at 17.5° C., and the mixture is stirred (1000 rpm) for 5 min without cooling, and then for 5 min while cooling using a cryostat at 0° C.

The microspheres are left to settle for a few minutes, and then filtered and rinsed with water so as to remove the residual PVA. They are then resuspended in 1 ml of water, frozen in liquid nitrogen and, finally, lyophilized.

Microscopic observation (see FIG. 1A) shows relatively spheroid, dense, oxaliplatin-loaded microspheres with varying sizes and surface appearances, the latter being linked to the oxaliplatin load.

Particle size analysis: average size 41.67 μm, median size 36.57 μm, standard deviation 30.51 μm, 60% of sizes distributed between 13.9 and 66.64 μm.

Production yield 85%. Encapsulation yield 81%. Degree of encapsulation 14%.

### EXAMPLE 2

Preparation of Microspheres of PLGA 25/50 of Molecular Mass 40 000 with a Degree of Encapsulation of Oxaliplatin of 31%.

(F3A)

307.9 mg of oxaliplatin powder are dispersed in 6 ml of ethyl acetate at 1° C. for 3 minutes with cooling in a cryostat at 0C. 498.5 mg of RG 503 polymer are added and dissolved with magnetic stirring at ambient temperature for 10 min, and then in a cryostat at 0° C. for 10 min. The temperature of the organic phase is 0.3° C.

The emulsion is formed, the ethyl acetate is extracted, and the microspheres are filtered, washed and lyophilized as described in example 1.

Microscopic observation shows relatively spheroid, dense, oxaliplatin-loaded microspheres having varying sizes (FIG. 1B). The oxaliplatin is visualized in the microspheres by reverse scattering (FIG. 1C).

Particle size analysis: average size 48.49 μm, median size 38.80 μm, standard deviation 40.66 μm, 60% of sizes distributed between 19.26 and 71.96 μm.

Production yield 90%. Encapsulation yield 83%. Degree of encapsulation 31%.

### EXAMPLE 3

Preparation of Microspheres of PLGA 25/50 of Molecular Mass 40 000 with a Degree of Encapsulation of Oxaliplatin of 20%.

176.8 mg of oxaliplatin powder are dispersed in 6 ml of ethyl acetate at 1° C. for 2 minutes with cooling in a cryostat at 0° C. 580 mg of RG 503 polymer are added and dissolved with magnetic stirring at ambient temperature for 10 min, and then in a cryostat at 0° C. for 10 min.

The emulsion is formed, the ethyl acetate is extracted, and the microspheres are filtered, washed and lyophilized in a manner similar to that described in example 1, with the main difference being that the temperature of the PVA solution is 8° C. and the temperature of the water for extraction is 34° C.

Microscopic observation shows relatively spheroid, dense, oxaliplatin-loaded microspheres having varying sizes of mainly between 5 and 100 μm.

Production yield 86%. Encapsulation yield 80%. Degree of encapsulation 20%.

The microspheres are passed through a sieve with a mesh size of 125 μm in order to remove the largest particles. They are then sterilized by irradiation with gamma rays at 25 kGy.

### EXAMPLE 4

Preparation of Microsphere of 90% PLGA 25/50 of Molecular Mass 40 000 and 10% PLA of Molecular Mass 2000 with a Degree of Encapsulation of Oxaliplatin of 20%.

154.0 mg of oxaliplatin powder are dispersed in 6 ml of ethyl acetate at 1° C. for 2 minutes with cooling in a cryostat at 0° C. 453 mg of RG 503 polymer and 54.7 mg of PLA50 oligomer are added and dissolved with magnetic stirring at ambient temperature for 10 min, and then in a cryostat at 0° C. for 10 min.

The emulsion is formed, the ethyl acetate is extracted, and the microspheres are filtered, washed and lyophilized in a manner similar to that described in example 1, with the main difference being that the temperature of the PVA solution is 6° C.

Microscopic observation shows relatively spheroid, dense, oxaliplatin-loaded microspheres having sizes of mainly between 5 and 100 μm.

Production yield 82%. Encapsulation yield 86%. Degree of encapsulation 20%.

The microspheres are passed through a sieve with a mesh size of 125 μm in order to remove the largest particles. They are then sterilized by irradiation with gamma rays at 25 kGy.

### EXAMPLE 5

Preparation of Microspheres of 80% PLGA 25/50 of Molecular Mass 40 000 and 20% PGLA 25/50 of Molecular Mass 10 000 with a Degree of Encapsulation of Oxaliplatin of 15%.

100.1 mg of oxaliplatin powder are dispersed in 6 ml of ethyl acetate at 1° C. for 3 minutes with cooling in a cryostat at 0° C. 397.8 mg of RG503 polymer and 106.8 mg of RG502 polymer are added and dissolved with magnetic stirring at ambient temperature for 10 min, and then in a cryostat at 0° C. for 10 min.

The emulsion is formed, the ethyl acetate is extracted, and the microspheres are filtered, washed and lyophilized in a manner similar to that described in example 1, with the main differences being that the temperature of the PVA solution is 12° C. and the water for extraction has a temperature of 30° C.

Microscopic observation shows spheroid, dense oxaliplatin-loaded microspheres having varying sizes, and few empty shells.

Particle size analysis: average size 48.02 μm, median size 37.82 μm, standard deviation 43.73 μm, 60% of sizes distributed between 20.10 and 66.40 μm.

Production yield 90%. Encapsulation yield 77%. Degree of encapsulation 15%.

### EXAMPLE 6

Oxaliplatin In Vitro Release Profiles for the Microspheres Obtained in Examples 1 to 5.

100 mg of microspheres obtained in example 1 to 5 are suspended in 1 1 of phosphate buffer, pH 7.4, with stirring at 100 rpm. Aliquot portions of the dissolving medium are withdrawn periodically and their platinum content is assayed using the ICP-MS (Inductively Coupled Plasma Mass Spectrometry) technique, while at the same time adding phosphate buffer to the dissolving medium in order to keep the volume of the dissolving medium constant. The method published by P. Allain et al., 1992, Biological mass spectrometry, 21, 141-143, is used here. The curve for release of oxaliplatin as a function of time is thus obtained for each of the microsphere batches.

The curves obtained for the microspheres obtained in examples 1, 2 and 5 are given in FIG. 2A.

These curves show that, for microspheres consisting of the same PGLA 25/50 polymer of molecular mass 40 000 (examples 1 and 2) the rate of release of the oxaliplatin and the initial burst are greater the higher the degree of encapsulation. After 1 200 hours, i.e. approximately 50 days, the degree of release of the oxaliplatin is 100% for the microspheres obtained in example 2 and 75% for the microspheres obtained in example 1.

The presence of a PGLA 25/50 polymer of molecular mass 10 000 in a proportion of 20% makes it possible, at a substantially equal degree of encapsulation (examples 1 and 5), to increase the rate of release of the oxaliplatin while at the same time decreasing the initial burst.

The curves obtained for the sterilized microspheres obtained in examples 3 and 4 are given in FIG. 2B.

Compared to the curves of FIG. 2A, the initial burst is greater and the first release plateau lasts for a shorter period of time. This is expected since it is known, in the art, that sterilization by irradiation by gamma rays has the effect of increasing the initial burst and decreasing the duration of the first release plateau.

These curves show that the presence of a PLA oligomer of molecular mass 2000 in a proportion of 10% makes it possible, at an equal degree of encapsulation (examples 3 and 4), to considerably accelerate the rate of release of the oxaliplatin.

### EXAMPLE 7

In Vivo Evaluation of the Antitumor Activity and of the Toxicity of the Microspheres of the Invention

Antitumor Activity on IGROV-1 Tumors After i.p. Administration

10⁷ viable IGROV-1 cells were inoculated i.p. into Nude mice. On day 15 after inoculation of tumor cells, the mice were given the substances tested by i.p. administration.

The mice were given a single i.p. administration of F3B microspheres (example 1) or F3A microspheres (example 2) at doses corresponding to 15 to 70 mg oxaliplatin base/kg. Free oxaliplatin was administered i.p. at doses of 6 and 15 mg base/kg. A group of mice was given 3 injections of free oxaliplatin at 6 mg base/kg per injection every 4 days (administration scheme q4d×3). Another group was given a single i.p. injection of free oxaliplatin at 15 mg/kg. The control group was given only a solution of 5% glucose or empty microspheres. Each group was made up of 5 mice. Isoflurane was used to anesthetize the animals before implantation of the tumor or sacrifice of the animals.

Antitumor Activity of PC-3 Human Prostate Tumors After i.t. Administration

10⁷ PC-3 cells were inoculated on the flank of male Nude mice. The treatment began from a tumor size of 210-266 mm³ (day 30).

The F3A and F3B microspheres were administered in a single i.t. injection of 15 or 30 mg of oxaliplatin/kg. The free oxaliplatin was administered by simple intravenous (i.v.) or i.t. injection at doses of 15 and 30 mg base/kg respectively. One group of mice was given 3 i.v. injections of free oxaliplatin at 6 mg base/kg per injection every 4 days (administration q4d×3). The volume of the test substances administered to the PC-3 tumors was equivalent to the tumor volume (1 μl of treatment per 1 mm³ of tumor).

The experiments were stopped when the size of the tumor reached 2 000 mm³ or when the period after the final treatment exceeded 60 days. Mice carrying progressive tumors were sacrificed. The mice were weighed and examined twice a week, and the weight of the sick animals was compared with the weight of three healthy mice (no tumor, no treatment, of the same batches). The animals were monitored for 98 days.

Results

Intraperitoneal Administration

As described in the biological evaluations, the oxaliplatin microspheres were tested on IGROV-1 tumor models, intraperitoneally, and on PC-3 tumor models, intratumorally. In the intraperitoneal context, the tumors were inoculated on day 0. The treatment was carried out 15 days after inoculation of the tumor cells, with F3A and F3B microspheres in comparison with free oxaliplatin administered either 3 times every four days, or as a single dose. The administration was carried out at varying doses and the result is shown in table 1. As can be seen in table 1, a significant antitumor activity was demonstrated for the F3B microspheres at a dose of 15 mg base/kg with oxaliplatin. The free oxaliplatin at a dose of 15 mg did not show any significant antitumor activity.

Intratumor Administration

The free oxaliplatin, injected intratumorally at a dose of 30 mg oxaliplatin base/kg, demonstrated considerable antitumor activity (T/C%<42%) but also acute toxicity (2 mice out of 5 died 4 to 7 days after treatment). The F3B and F3A microspheres (at doses of 30 and 15 mg base/kg) showed significant antitumor activity (T/C%<42%) with no acute toxicity. The antitumor activity was observed later with oxaliplatin encapsulated in microspheres compared to that observed with the free oxaliplatin administered i.t. at doses of 30 mg base/kg, but the inhibition of tumor growth was identical. In conclusion, the encapsulation of oxaliplatin in microspheres increases oxaliplatin tolerance. The tolerance depends on the rate of release and on the oxaliplatin load in the microspheres.

TABLE 1
Effects of the treatment after i.p. injection
of F3B or F3A microspheres or of free oxaliplatin
in Nude mice carrying IGROV-1 xenografts

| | Dose of | | |
|---|---|---|---|
| | oxaliplatin | | Survival |
| Treatment | (mg/kg) | T/C %⁽²⁾ | at D98 |
| Oxaliplatin | 6⁽¹⁾ | 85.71 | 0/5 |
| Oxaliplatin | 15 | 102.38 | 1/5 |
| Example 1. F3B | 15 | 135.71 | 1/5 |
| Example 1. F3B | 70 | 111.90 | 1/5 |
| Example 2. F3A | 15 | 111.90 | 0/5 |
| Example 2. F3A | 70 | 61.90 | 0/5 |

⁽¹⁾dose administered 3 times every four days
⁽²⁾T/C % = ratio of the median of survival time of the treated animals to the median of survival time of the untreated control group × 100

TABLE 2
Effects of the treatment by i.t. injection of F3B or F3A microspheres
or free oxaliplatin in Nude mice carrying PC-3 zenografts

| | Dose of | |
|---|---|---|
| | oxaliplatin in | |
| | mg/kg (route of | |
| Treatment | administration) | T/C %⁽²⁾ |
| Oxaliplatin | 6⁽¹⁾ (i.v.) | 80.41 |
| Oxaliplatin | 15 (i.v.) | 55.47 |
| Oxaliplatin | 30 (i.t.) | 5.24 |
| Example 1. F3B | 15 (i.t.) | 44.38 |
| Example 1. F3B | 30 (i.t.) | 29.39 |
| Example 2. F3A | 15 (i.t.) | 46.17 |
| Example 2. F3A | 30 (i.t.) | 36.71 |

⁽¹⁾dose administered three times every four days
⁽²⁾T/C % = ratio of the median of average survival time of the treated animals to the median of average survival time of the untreated control group × 100.

1. A method for preparing, by solvent extraction, poly(D,L-lactide-co-glycolide)/poly(D,L-lactide) (PLGA/PLA)-type polymer microspheres encapsulating a water-soluble therapeutic substance which is not soluble in ethyl acetate, comprising the following steps:
(a) preparing an organic phase comprising dissolved polymer and particles of the therapeutic substance in suspension in ethyl acetate, and cooling this organic phase so as to increase its viscosity,
(b) adding to this organic phase a cold aqueous solution of surfactant with vigorous stirring so as to form an emulsion,
(c) extracting the ethyl acetate by adding hot water, with stirring to the emulsion kept at low temperature, which induces microsphere formation,
(d) filtering the heterogeneous mixture obtained in (c) so as to retain the microspheres formed and rinsing so as to remove the residual surfactant and, optionally,
(d) lyophilizing the microspheres.

2. The method as claimed in claim 1, characterized in that the organic phase prepared in step (a) is cooled to a temperature of 8-0° C., preferably 4-0° C.

3. The method as claimed in either of the preceding claims, characterized in that the temperature of the cold aqueous solution of surfactant added in step (b) is from 6 to −20° C., preferably from 3 to 0° C.

4. The method as claimed in one of the preceding claims, characterized in that, during step (c), the emulsion is maintained at a temperature below 8° C., preferably below 5° C.

5. The method as claimed in one of the preceding claims, characterized in that the hot water added during step (c) has a temperature of from 26 to 40° C., preferably 28 to 35° C.

6. The method as claimed in one of the preceding claims, characterized in that the ratio of the volume of ethyl acetate used to prepare the organic phase to the volume of cold aqueous solution of surfactant is 0.10-0.20, preferably 0.12-0.18.

7. The method as claimed in one of the preceding claims, characterized in that the volume of ethyl ethyl acetate used to prepare the organic phase is 8-16 ml, preferably 10-14 ml per g of polymer.

8. The method as claimed in one of the preceding claims, characterized in that the volume of water added during step (c) is at least approximately 10 times, preferably at least 20 times, that of ethyl acetate.

9. The method as claimed in one of the preceding claims, characterized in that the addition of hot water during step (c) is carried out gradually.

10. The method as claimed in one of the preceding claims, characterized in that the surfactant is a nonionic surfactant, for example a polyvinyl alcohol.

11. The method as claimed in one of the preceding claims, characterized in that the PGLA has a molecular weight of less than 50 000, preferably of between 10 000 and 40 000.

12. The method as claimed in one of the preceding claims, characterized in that the PGLA is 25/50.

13. The method as claimed in one of the preceding claims, characterized in that the therapeutic substance is oxaliplatin.

14. A PLGA/PLA microsphere encapsulating oxaliplatin which can be obtained using the method as claimed in one of the preceding claims.

15. A PLGA/PLA miscrosphere encapsulating oxaliplatin with a degree of encapsulation of at least 10%, preferably of 15-40%, in particular as claimed in claim 14.

16. The microsphere as claimed in either of claims 14 and 15, characterized in that it has an average size of 20-100 μm, preferably 30-70 μm.

17. The microsphere as claimed in one of claims 14 to 16, for use as a medicinal product.

18. A pharmaceutical formulation for intraperitoneal, intratumor or intrapleural administration, comprising microspheres as claimed in one of claims 14 to 16, in a pharmaceutically acceptable vehicle.
